(19) United States
(12) Patent Application Publication
(10) Pub. No.: US 2020/0048198 A1
(43) Pub. Date: Feb. 13, 2020

(54) MODIFIED HISTONE DEACETYLASE INHIBITORS AND USES THEREOF

(71) Applicants: The Regents of the University of California, Oakland, CA (US); Vybyl Holdings. Inc., Woodridge, IL (US)

(72) Inventors: Nathan C Gianneschi, San Diego, CA (US); Paul A. Bertin, Woodridge, IL (US)

(21) Appl. No.: 16/492,656

(22) PCT Filed: Mar. 21, 2018
(86) PCT No.: PCT/US18/23581

§ 371 Date: Sep. 10, 2019

Related U.S. Application Data
(60) Provisional application No. 62/475,187, filed on Mar. 22, 2017.

(51) Int. Cl. C07D 209/14; C07C 271/60; C07C 233/07; A61K 38/38

(52) U.S. Cl. CPC C07D 209/14; C07C 271/60; A61K 45/06; A61K 38/385; C07C 233/07

(57) ABSTRACT

The present disclosure generally provides compounds useful for treating cancer. In some aspects, the disclosure provides small-molecule histone deacetylase inhibitors (HDIs) that are chemically modified to have one or more moieties that include hydrophobic portions. In some aspects, the disclosure provides compositions that include such modified HDIs and a protein, such as albumin or albumin mimetics. Further, the disclosure provides various uses of these compounds and compositions.

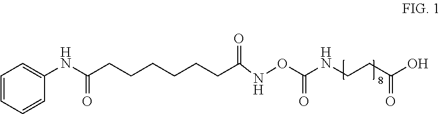

### CROSS-REFERENCE TO RELATED APPLICATIONS

The present application claims the benefit of priority to U.S. Provisional Application No. 62/475,187, filed Mar. 22, 2017, which is hereby incorporated by reference as though set forth herein in its entirety.

### TECHNICAL FIELD

The present disclosure generally provides compounds useful for treating cancer. In some aspects, the disclosure provides small-molecule histone deacetylase inhibitors (HDIs) that are chemically modified to have one or more moieties that include hydrophobic portions. In some aspects, the disclosure provides compositions that include such modified HDIs and a protein, such as albumin or albumin mimetics. Further, the disclosure provides various uses of these compounds and compositions.

### DESCRIPTION OF RELATED ART

Cancer refers to a group of diseases characterized by the formation of malignant tumors or neoplasms, which involve abnormal cell growth and have the potential to invade adjacent tissue and spread to other parts of the body. There are more than 14 million new diagnoses of cancer annually. Moreover, cancer accounts for more than 8 million deaths each year, which is about 15% of all deaths worldwide. In developed countries, cancer accounts for an even higher percentage of deaths.

Therapies for cancer have improved significantly over the years. In particular, an increasing number of cytotoxic agents have been discovered. These agents generally work by killing the cancer cells. But cytotoxic agents can be harmful to normal cells as well. Therefore, subjects undergoing treatment with such agents often suffer certain side-effects from the treatment. In some cases, the side-effects pose such a substantial risk that it may be necessary to administer very limited quantities of cytotoxic agents. So, while there is a general desire to discover increasingly toxic chemotherapeutic agents, it is also desirable to develop new means of directing those compounds selectively to cancer cells and away from normal cells.

Various strategies have been used to assist in directing chemotherapeutic agents selectively to cancer cells. For example, HDIs represent a class of compounds that are useful in treating certain cancers. HDIs interfere with the coiling and uncoiling of DNA around histone compounds in the interior of a cancer cell. As a result, HDIs tend to arrest proliferation of the cells, and eventually induce cell cycle arrest, cell differentiation, and eventual apoptosis. Even so, HDIs can have a number of other effects following administration. In fact, HDIs have long been used to treat various psychiatric and neurological conditions. Thus, the utility of HDIs for treating cancer can be limited by their tendency to have off-target effects. HDIs also have a tendency to be metabolized rapidly, which prevents their dosing at the kinds of concentrations that may be desirable for treatment of certain kinds of cancer. Therefore, there is a continuing need to discover new ways of selectively directing HDIs to cancer cells and tumors, so as to reduce off-target effects and to improve their pharmacokinetic profile.

### SUMMARY

The present disclosure provides compounds and compositions that can deliver increasingly high quantities of HDIs to cancer cells (e.g., in a solid tumor) with reduced off-target side effects and improved pharmacokinetic lifetime. In some embodiments, the compounds are prodrugs of small-molecule HDIs, such that the prodrug permits improved delivery of the HDI to a solid tumor in a mammal. The disclosure also provides methods and uses of those compounds and compositions for the treatment of cancer, including uses as an adjuvant therapy to various immunotherapy-based treatments.

In a first aspect, the disclosure provides compounds of formula (I):

A¹-X¹—X²-A² (I)

wherein: A¹ is an organic group, or is a hydrophilic group, or a hydrogen atom; A² is a histone deacetylase inhibiting moiety; X¹ is a hydrophobic group; and X² is a direct bond, an organic group, or a heteroatom group selected from the group consisting of —O—, —S—, —S(═O)—, —S(═O)₂—, —S—S—, —N═, ═N—, —N(H)—, —N═N—N(H)—, —N(H)—N═N—, —N(OH)—, or —N(═O)—. In some embodiments, A¹ is a hydrophilic group, such as a carboxylic acid group (—COOH) or a pharmaceutically acceptable salt thereof. In some embodiments, the histone deacetylase inhibiting moiety is a vorinostat moiety, a belinostat moiety, or a panobinostat moiety. In some embodiments, the hydrophobic group is a C₁₂₋₂₂ hydrocarbylene group, which is optionally substituted. In some embodiments, X² is an organic group, such as a carbonyl group, i.e., —C(═O)—.

In a second aspect, the disclosure provides compositions (e.g., pharmaceutical compositions) that include: a compound of any embodiments of the first aspect; and a protein. In some embodiments, the protein is an albumin or an albumin mimetic.

In a third aspect, the disclosure provides compositions (e.g., pharmaceutical compositions) that include: a compound of any embodiments of the first aspect; a protein, wherein the protein is an albumin or an albumin mimetic; and a carrier, which includes water; wherein the compound and the protein are non-covalently associated with each other; and wherein the compound and the protein are solvated by the carrier.

In a fourth aspect, the disclosure provides methods of treating cancer, which include administering to a subject a compound or composition of any embodiments of any of the foregoing aspects. In some further embodiments thereof, the disclosure provides methods of treating cancer that include administering to a subject one or more immunotherapy agents.

In a fifth aspect, the disclosure provides methods of inducing apoptosis in a cancer cell, which include contacting the cancer cell with a compound or composition of any embodiments of any of the first through the third aspects. In some further embodiments thereof, the disclosure provides methods of inducing apoptosis in a cancer cell that include contacting the cancer cell with one or more immunotherapy agents.

In a sixth aspect, the disclosure provides methods for inhibiting growth of a cancerous tumor, which includes contacting the cancerous tumor with a compound of any embodiments of the first aspect. In some further embodiments thereof, the disclosure provides methods of inhibiting growth of a cancerous tumor that include contacting the cancerous tumor with one or more immunotherapy agents.

In a seventh aspect, the disclosure provides uses of a compound or composition of any embodiments of any of the first through the third aspects as a medicament.

In an eighth aspect, the disclosure provides uses of a compound or composition of any embodiments of any of the first through the third aspects for treating cancer. In some further embodiments thereof, the disclosure provides uses that include use in combination with one or more immunotherapy agents.

In a ninth aspect, the disclosure provides uses of a compound or composition of any embodiments of any of the first through the third aspects in the manufacture of a medicament.

In a tenth aspect, the disclosure provides uses of a compound or composition of any embodiments of any of the first through the third aspects in the manufacture of a medicament for treating cancer.

In an eleventh aspect, the disclosure provides methods of making compounds of the first and second aspects and compositions of the third and fourth aspects.

Further aspects and embodiments are provided in the drawings, the detailed description, the claims, and the abstract.

### BRIEF DESCRIPTION OF DRAWINGS

The following drawings are provided for purposes of illustrating various embodiments of the compounds, compositions, methods, and uses disclosed herein. The drawings are provided for illustrative purposes only, and are not intended to describe any preferred compounds or compositions or any preferred methods or uses, or to serve as a source of any limitations on the scope of the claimed inventions.

FIG. 1 shows a non-limiting example of a compound of formula (I), where the compound includes a vorinostat moiety, which is modified to include a long-chain dibasic acid moiety.

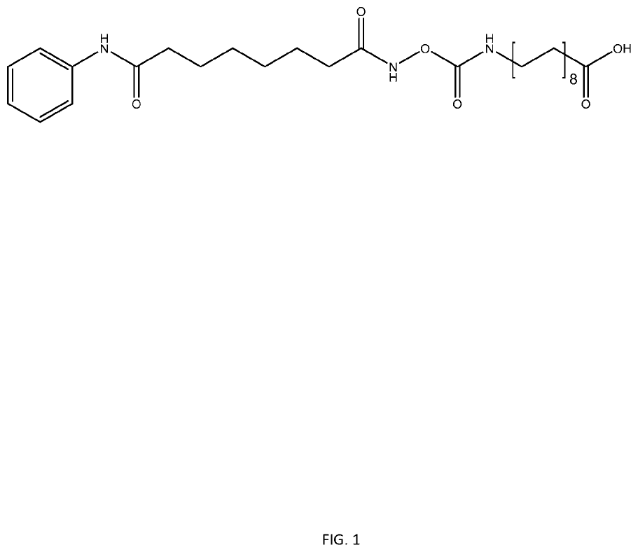

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “hydrocarbon” refers to an organic group composed of carbon and hydrogen, which can be saturated or unsaturated, and can include aromatic groups. The term “hydrocarbyl” refers to a monovalent or polyvalent (e.g., divalent or higher) hydrocarbon moiety. In some cases, a divalent hydrocarbyl group is referred to as a “hydrocarbylene” group.

As used herein, “alkyl” refers to a straight or branched chain saturated hydrocarbon having 1 to 30 carbon atoms, which may be optionally substituted, as herein further described, with multiple degrees of substitution being allowed. Examples of “alkyl,” as used herein, include, but are not limited to, methyl, ethyl, n-propyl, isopropyl, isobutyl, n-butyl, sec-butyl, tert-butyl, isopentyl, n-pentyl, neopentyl, n-hexyl, and 2-ethylhexyl. In some instances, the “alkyl” group can be divalent, in which case, the group can alternatively be referred to as an “alkylene” group. Also, in some instances, one or more of the carbon atoms in the alkyl or alkylene group can be replaced by a heteroatom (e.g., selected from nitrogen, oxygen, or sulfur, including N-oxides, sulfur oxides, sulfur dioxides, and carbonyl groups, where feasible), and is referred to as a “heteroalkyl” or “heteroalkylene” group, respectively. Non-limiting examples include “oxyalkyl” or “oxyalkylene” groups, which refer to groups where a carbon atom in the alkyl or alkylene group is replaced by oxygen. Non-limiting examples of oxyalkyl or oxyalkylene groups include alkyl or alkylene chains that contain a carbonyl group, and also alkoxylates, polyalkylene oxides, and the like.

The number of carbon atoms in any group or compound can be represented by the terms. Thus, “CZ” refers to a group of compound having z carbon atoms, and “C_{x-y}”, refers to a group or compound containing from x to y, inclusive, carbon atoms. For example, “C₁₋₆ alkyl” represents an alkyl group having from 1 to 6 carbon atoms and, for example, includes, but is not limited to, methyl, ethyl, n-propyl, isopropyl, isobutyl, n-butyl, sec-butyl, tert-butyl, isopentyl, n-pentyl, neopentyl, and n-hexyl. The same logic applies to other types of functional groups, defined below.

As used herein, “alkenyl” refers to a straight or branched chain non-aromatic hydrocarbon having 2 to 30 carbon atoms and having one or more carbon-carbon double bonds, which may be optionally substituted, as herein further described, with multiple degrees of substitution being allowed. Examples of “alkenyl,” as used herein, include, but are not limited to, ethenyl, 2-propenyl, 2-butenyl, and 3-butenyl. In some instances, the “alkenyl” group can be divalent, in which case the group can alternatively be referred to as an “alkenylene” group. Also, in some instances, one or more of the carbon atoms in the alkenyl or alkenylene group can be replaced by a heteroatom (e.g., selected from nitrogen, oxygen, or sulfur, including N-oxides, sulfur oxides, sulfur dioxides, and carbonyl groups, where feasible), and is referred to as a “heteroalkenyl” or “heteroalkenylene” group, respectively.

As used herein, “cycloalkyl” refers to an aliphatic saturated or unsaturated hydrocarbon ring system having 3 to 20 carbon atoms, which may be optionally substituted, as herein further described, with multiple degrees of substitution being allowed. In some embodiments, the term refers only to saturated hydrocarbon ring systems, substituted as herein further described. Examples of “cycloalkyl,” as used herein, include, but are not limited to, cyclopropyl, cyclobutyl, cyclopentyl, cyclohexyl, cyclohexenyl, cycloheptyl, cyclooctyl, adamantyl, and the like. In some instances, the “cycloalkyl” group can be divalent, in which case the group can alternatively be referred to as a “cycloalkylene” group. Cycloalkyl and cycloalkylene groups can also be referred to herein as “carbocyclic rings.” Also, in some instances, one or more of the carbon atoms in the cycloalkyl or cycloalkylene group can be replaced by a heteroatom (e.g., selected independently from nitrogen, oxygen, silicon, or sulfur, including N-oxides, sulfur oxides, and sulfur dioxides, where feasible), and is referred to as a “heterocyclyl” or “heterocyclylene” group, respectively. The term “heterocyclic ring” can also be used interchangeably with either of these terms. In some embodiments, the cycloalkyl and heterocyclyl groups are fully saturated. In some other embodiments, the cycloalkyl and heterocyclyl groups can contain one or more carbon-carbon double bonds.

As used herein, “halogen,” “halogen atom,” or “halo” refer to a fluorine, chlorine, bromine, or iodine atom. In some embodiments, the terms refer to a fluorine or chlorine atom.

As used herein, the terms “organic group,” “organic moiety,” or “organic residue” refer to a monovalent or polyvalent functional group having at least one carbon atom, which optionally contains one or more additional atoms selected from the group consisting of hydrogen atoms, halogen atoms, nitrogen atoms, oxygen atoms, phosphorus atoms, and sulfur atoms, and which does not include covalently bound metal or semi-metal atoms. In some embodiments, these terms can include metal salts of organic groups, such as alkali metal or alkaline earth metal salts of organic anions.

As used herein, the term “pharmacophore” refers to a type of organic functional group. Standard pharmacophores are hydrophobic pharmacophores, hydrogen-bond donating pharmacophores, hydrogen-bond accepting pharmacophores, positive ionizable pharmacophores, and negative ionizable pharmacophores. The classification of organic functional groups within a compound is carried out according to standard classification systems known in the art.

As used herein, the terms “hydrophobic group,” “hydrophobic moiety,” or “hydrophobic residue” refer to an organic group that consists essentially of hydrophobic pharmacophores. In some embodiments, the terms refer to an organic group that consists of hydrophobic pharmacophores.

As used herein, the terms “hydrophilic group,” “hydrophilic moiety,” or “hydrophilic residue” refer to an organic group that comprises one pharmacophores selected from the group consisting of hydrogen bond donors, hydrogen bond acceptors, negative ionizable groups, or positive ionizable groups. In some embodiments, the terms refer to an organic group that consist essentially of pharmacophores selected from the group consisting of hydrogen bond donors, hydrogen bond acceptors, negative ionizable groups, or positive ionizable groups.

As used herein, the term “histone deacetylase inhibiting moiety” refers to a histone deacetylase inhibiting compound, or a pharmaceutically acceptable salt thereof, where an atom or a group of atoms is absent, thereby creating a monovalent or polyvalent moiety. In some embodiments, for example, a hydrogen atom is absent, thereby creating a monovalent moiety. In some other embodiments, a functional group, such as an —OH moiety, an —NH₂ moiety, or a —COOH, moiety is absent. One non-limiting example of such a “histone deacetylase inhibiting moiety,” (in this case, a “vorinostat moiety”) is the moiety of the following formula:

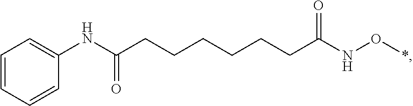

where a hydrogen atom is absent to create a monovalent moiety that, within a compound, bonds to the rest of the molecule through the remaining oxygen atom. Note that the term “histone deacetylase inhibiting moiety” is not limited to any particular procedure for making such compounds or moieties.

Various methods of drawing chemical structures are used herein. In some instances, the bond line-structure method is used to depict chemical compounds or moieties. In the line-structure method, the lines represent chemical bonds, and the carbon atoms are not explicitly shown (but are implied by the intersection of the lines). The hydrogen atoms are also not explicitly shown, except in instances where they are attached to heteroatoms. Heteroatoms, however, are explicitly shown. Thus, using that methodology, the structures shown below are for 2-methylpropane, 1-methoxypropane, and 1-propanol:

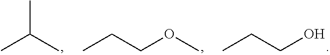

In that methodology, aromatic rings are typically represented merely by one of the contributing resonance structures. Thus, the following structures are for benzene, pyridine, and pyrrole:

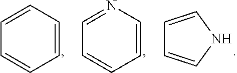

As used herein, a “protein binding moiety” is a moiety that binds non-covalently to one or more sites on a protein with a binding constant (K_{b}) of at least 100 M⁻¹ in water at 25° C.

As used herein, “amino acid” refers to a compound having the structure H₂N—R^{x}—COOH, where R^{x} is an organic group, and where the NH₂ may optionally combine with R^{x} (e.g., as in the case of proline). The term includes any known amino acids, including, but not limited to, alpha amino acids, beta amino acids, gamma amino acids, delta amino acids, and the like. In some embodiments, the term can refer to alpha amino acids.

As used herein, “hydroxy acid” refers to a compound having the structure HO—R^{y}—COOH, where R^{y} is an organic group. Non-limiting examples include glycolic acid, lactic acid, and caprolactone.

As used herein, “alkanol amine” refers to a compound having the structure HO—R^{z}—NH₂, where R^{z} is an optionally substituted alkylene group. Non-limiting examples include ethanol amine.

As used herein, “administer” or “administering” means to introduce, such as to introduce to a subject a compound or composition. The term is not limited to any specific mode of delivery, and can include, for example, subcutaneous delivery, intravenous delivery, intramuscular delivery, intracisternal delivery, delivery by infusion techniques, transdermal delivery, oral delivery, nasal delivery, and rectal delivery. Furthermore, depending on the mode of delivery, the administering can be carried out by various individuals, including, for example, a health-care professional (e.g., physician, nurse, etc.), a pharmacist, or the subject (i.e., self-administration).

As used herein, “treat” or “treating” or “treatment” can refer to one or more of: delaying the progress of a disease, disorder, or condition; controlling a disease, disorder, or condition; ameliorating one or more symptoms characteristic of a disease, disorder, or condition; or delaying the recurrence of a disease, disorder, or condition, or characteristic symptoms thereof, depending on the nature of the disease, disorder, or condition and its characteristic symptoms.

As used herein, “subject” refers to any mammal such as, but not limited to, humans, horses, cows, sheep, pigs, mice, rats, dogs, cats, and primates such as chimpanzees, gorillas, and rhesus monkeys. In some embodiments, the “subject” is a human. In some such embodiments, the “subject” is a human who exhibits one or more symptoms characteristic of a disease, disorder, or condition. The term “subject” does not require one to have any particular status with respect to a hospital, clinic, or research facility (e.g., as an admitted patient, a study participant, or the like).

As used herein, the term “compound” includes free acids, free bases, and salts thereof.

As used herein, the term “pharmaceutical composition” is used to denote a composition that may be administered to a mammalian host, e.g., orally, topically, parenterally, by inhalation spray, or rectally, in unit dosage formulations containing conventional non-toxic carriers, diluents, adjuvants, vehicles and the like. The term “parenteral” as used herein, includes subcutaneous injections, intravenous, intramuscular, intracisternal injection, or by infusion techniques.

Also included within the scope of the disclosure are the individual enantiomers of the compounds represented by Formula (I) or pharmaceutically acceptable salts thereof, as well as any wholly or partially racemic mixtures thereof. The disclosure also covers the individual enantiomers of the compounds represented by Formula (I) or pharmaceutically acceptable salts thereof, as well as mixtures with diastereoisomers thereof in which one or more stereocenters are inverted. Unless otherwise stated, structures depicted herein are also meant to include compounds which differ only in the presence of one or more isotopically enriched atoms. For example, compounds having the present structure, except for the replacement of a hydrogen atom by a deuterium or tritium, or the replacement of a carbon atom by a ¹³C- or ¹⁴C-enriched carbon are within the scope of the disclosure.

As used herein, “mix” or “mixed” or “mixture” refers broadly to any combining of two or more compositions. The two or more compositions need not have the same physical state; thus, solids can be “mixed” with liquids, e.g., to form a slurry, suspension, or solution. Further, these terms do not require any degree of homogeneity or uniformity of composition. This, such “mixtures” can be homogeneous or heterogeneous, or can be uniform or non-uniform. Further, the terms do not require the use of any particular equipment to carry out the mixing, such as an industrial mixer.

As used herein, “optionally” means that the subsequently described event(s) may or may not occur. In some embodiments, the optional event does not occur. In some other embodiments, the optional event does occur one or more times.

As used herein, “substituted” refers to substitution of one or more hydrogen atoms of the designated moiety with the named substituent or substituents, multiple degrees of substitution being allowed unless otherwise stated, provided that the substitution results in a stable or chemically feasible compound. A stable compound or chemically feasible compound is one in which the chemical structure is not substantially altered when kept at a temperature from about −80° C. to about +40° C., in the absence of moisture or other chemically reactive conditions, for at least a week. As used herein, the phrases “substituted with one or more . . . ” or “substituted one or more times . . . ” refer to a number of substituents that equals from one to the maximum number of substituents possible based on the number of available bonding sites, provided that the above conditions of stability and chemical feasibility are met.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present. As used herein, the phrases “consist essentially of” “consists essentially of” and “consisting essentially of” refer to groups that are open, but which only includes additional unnamed members that would not materially affect the basic characteristics of the claimed subject matter.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

As used herein, the various functional groups represented will be understood to have a point of attachment at the functional group having the hyphen or dash (-) or a dash used in combination with an asterisk (*). In other words, in the case of —CH₂CH₂CH₃ or *—CH₂CH₂CH₃, it will be understood that the point of attachment is the CH₂ group at the far left. If a group is recited without an asterisk or a dash, then the attachment point is indicated by the plain and ordinary meaning of the recited group.

As used herein, multi-atom bivalent species are to be read from left to right. For example, if the specification or claims recite A-D-E and D is defined as —OC(O)—, the resulting group with D replaced is: A-OC(O)-E and not A-C(O)O-E.

Other terms are defined in other portions of this description, even though not included in this subsection.

### Modified Histone Deacetylase Inhibitors

In at least one aspect, the disclosure provides compounds of formula (I):

A¹-X¹—X²-A² (I)

wherein: A¹ is a hydrophilic group or a hydrogen atom, or is an organic group; A² is a histone deacetylase inhibiting moiety; X¹ is a hydrophobic group; and X² is a direct bond, an organic group, or a group selected from the group consisting of —O—, —S—, —S(═O)—, —S(═O)₂—, —S—S—, —N═, ═N—, —N(H)—, —N═N—N(H)—, —N(H)—N═N—, —N(OH)—, or —N(═O)—.

In some embodiments, A¹ is an organic group. A¹ can contain any suitable number of carbon atoms. In some embodiments, for example, A¹ contains from 1 to 100 carbon atoms, or from 1 to 50 carbon atoms, or from 1 to 25 carbon atoms, or from 1 to 10 carbon atoms, or from 1 to 6 carbon atoms. A¹ can also contain one or more heteroatoms, such as nitrogen, oxygen, sulfur, or phosphorus.

In some embodiments according to any of the foregoing embodiments, A¹ is a hydrophilic group or moiety. Non-limiting examples of a hydrophilic group include, but are not limited to, a carboxylic acid moiety, an ester moiety, an amide moiety, a urea moiety, an amine moiety, an ether moiety, an alcohol moiety, a thioether moiety, a thiol moiety, a ketone moiety, an aldehyde moiety, a sulfate moiety, a thiosulfate moiety, a sulfite moiety, a thiosulfite moiety, a phosphate moiety, a phosphonate moiety, a phosphinate moiety, a phosphite moiety, a borate moiety, or a boronate moiety.

In some embodiments of any of the aforementioned embodiments, A¹ is selected from the group consisting of a carboxylic acid group (—COOH), a carboxylate anion (—COO), or a carboxylate ester (—COOR^{a}, where R^{a} is an organic group such as an alkyl or alkoxylate group). In some such embodiments, A¹ is a carboxylic acid group. In some such embodiments, A¹ is a carboxylate ester group.

In some other embodiments of any of the aforementioned embodiments, A¹ is a hydrogen atom. In some other embodiments of any of the aforementioned embodiments, A¹ is a hydroxyl (—OH) group.

In any of the aforementioned embodiments, X¹ can be a hydrophobic group having any suitable number of carbon atoms. In some embodiments, for example, X¹ contains from 1 to 100 carbon atoms, or from 1 to 50 carbon atoms, or from 1 to 25 carbon atoms.

In some embodiments of any of the aforementioned embodiments, X¹ is C₈₋₃₀ hydrocarbylene, which is optionally substituted. In some further embodiments, X¹ is C₁₂₋₂₂ hydrocarbylene, which is optionally substituted. In some further embodiments, X¹ is C₁₂₋₂₂ alkylene. In some further embodiments, X¹ is —(CH₂)₁₂—, —(CH₂)₁₄—, —(CH₂)₁₆—, —(CH₂)₁₈—, —(CH₂)₂₀—, or —(CH₂)₂₂—. In some other embodiments, X¹ is —(CH₂)₁₆—. In some further embodiments, X¹ is C₁₂₋₂₂ alkenylene. In some further such embodiments, X¹ is —(CH₂)₇—CH═CH—(CH₂)₇—.

In some further embodiments of any of the aforementioned embodiments, X¹ is C₁₂₋₂₂ hydrocarbylene, which is optionally substituted. In some such embodiments, X¹ is C₁₂₋₂₂ hydrocarbylene. In some further such embodiments, X¹ is C₁₄₋₂₂ hydrocarbylene. In some further such embodiments, X¹ is C₁₆₋₂₂ hydrocarbylene. In some embodiments of any of the aforementioned embodiments, X¹ is C₁₂₋₂₂ hydrocarbylene, wherein A¹ and X² (or, if X² is a direct bond, A²) are separated from each other by at least 6, or by at least 8, or by at least 10, or by at least 12, or by at least 14, carbon atoms. In some further such embodiments, X¹ is C₁₄₋₂₂ hydrocarbylene, wherein A¹ and X² (or, if X² is a direct bond, A²) are separated from each other by at least 6, or by at least 8, or by at least 10, or by at least 12, or by at least 14, carbon atoms. In some further such embodiments, X¹ is C₁₆₋₂₂ hydrocarbylene, wherein A¹ and X² (or, if X² is a direct bond, A²) are separated from each other by at least 6, or by at least 8, or by at least 10, or by at least 12, or by at least 14, carbon atoms. In some further embodiments of any of the aforementioned embodiments, X¹ is C₁₂₋₂₂ straight-chain alkylene, or C₁₄₋₂₂ straight-chain alkylene, or C₁₆₋₂₂ straight-chain alkylene. In some further embodiments of any of the aforementioned embodiments, X¹ is C₁₂₋₂₂ straight-chain alkenylene, or C₁₄₋₂₂ straight-chain alkenylene, or C₁₆₋₂₂ straight-chain alkenylene.

In some embodiments of any of the aforementioned embodiments, X² is a direct bond. In some other embodiments of any of the aforementioned embodiments, X² is an organic group. In some embodiments, X² is a hydrophilic group. In some embodiments, X² is a heteroalkylene group.

In any of the aforementioned embodiments where X² is an organic group, X² can contain any suitable number of carbon atoms. In some embodiments, for example, X² contains from 1 to 100 carbon atoms, or from 1 to 50 carbon atoms, or from 1 to 25 carbon atoms, or from 1 to 10 carbon atoms, or from 1 to 6 carbon atoms.

In any of the aforementioned embodiments where X² is a heteroalkylene group, X² can contain any suitable number of carbon atoms. In some embodiments, for example, X² contains from 1 to 100 carbon atoms, or from 1 to 50 carbon atoms, or from 1 to 25 carbon atoms, or from 1 to 10 carbon atoms, or from 1 to 6 carbon atoms.

In some of the aforementioned embodiments, X² can contain certain groups. Some non-limiting examples of such groups that X² can contain are polyalkylene oxide groups, such as polyethylene glycol (PEG) and various polypeptide chains.

In some embodiments, X² is an organic group selected from the group consisting of —C(═O)—, —C(H)═C(H)—, —C(═O)—O—, —O—C(═O)—, —C(═O)—NH—, —NH—C(═O)—, —NH—C(═O)—O—, —O—(C═O)—NH—, —O—C(═O)—O—, —C(═N—NH₂)—, —C(═N—R^{b})— (where R^{b} is a hydrogen atom or an alkyl group), —C(═N—OH)—, —NH—C(═O)—NH—, —NH—C(═S)—NH—, —NH—C(═S)—O—, —O—C(═S)—NH—, —NH—C(═O)—S—, —S—C(═O)—NH—, —NH—C(═S)—S—, —S—C(═S)—NH—, and the cyclic structures shown below:

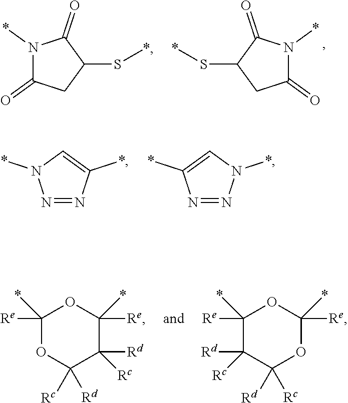

where R^{c}, R^{d}, and R^{e} are, independently at each occurrence, a hydrogen atom or C₁₋₁₀ alkyl. In some further embodiments, X² is —C(═O)—.

In some embodiments, X² is a group selected from the group consisting of —O—, —S—, —S(═O)—, —S(═O)₂—, —S—S—, —N═, ═N—, —N(H)—, —N═N—N(H)—, —N(H)—N═N—, —N(OH)—, and —N(O)—.

In some embodiments, X² comprises one or more moieties selected from the group consisting of: —C(═O)—, —O—C(═O)—, —NH—C(═O)—, one or more moieties formed from a alkylene glycols, one or more units formed from alkanol amines, one or more units formed from amino acids, and one or more units formed from hydroxyl acids. Thus, in some embodiments, X² comprises one or more moieties formed from alkylene glycols, such as a short poly(ethylene glycol) chain having 1 to 25 ethylene glycol units. In some embodiments, X² comprises one or more moieties formed from amino acids, such as an oligopeptide chain having 1 to 25 amino acid units. In some embodiments, X² comprises one or more moieties formed from hydroxy acids, such as moieties formed from glycolic acid, lactic acid, or caprolactone. In some embodiments, X² comprises a combination of a poly(ethylene glycol) chain having 1 to 25 ethylene glycol units and an oligopeptide having 1 to 25 amino acid units, and optionally one or more units formed from hydroxy acids.

In any of the above embodiments, the selection of X² will depend on the type of functional group through which it is linked to the histone deacetylase inhibiting moiety, so as to avoid making compounds that are chemically unstable or impossible. The skilled artisan will be able to select combinations of X² and A² that result in chemically stable compounds, which are compounds in which the chemical structure is not substantially altered when kept at a temperature from about −80° C. to about +40° C., in the absence of moisture or other chemically reactive conditions, for at least a week.

In the above embodiments, A² can be any suitable histone deacetylase inhibiting moiety. In some embodiments, the histone deacetylase inhibiting moiety is a small-molecule histone deacetylase inhibiting moiety, such as a histone deacetylase inhibiting moiety having a molecular weight of or no more than 1600 Da, or no more than 1500 Da, or no more than 1400 Da, or no more than 1300 Da, no more than 1200 Da, or no more than 1100 Da, or no more than 1000 Da, or no more than 900 Da. Such histone deacetylase inhibiting moieties can be organic moieties, or can also be moieties that contain inorganic atoms. In some embodiments, however, the histone deacetylase inhibiting moiety is an organic moiety.

In some embodiments of any of the aforementioned embodiments, the histone deacetylase inhibiting moiety is a moiety selected from the group consisting of: a vorinostat moiety, a romidepsin moiety, a chidamide moiety, a panobinostat moiety, a belinostat moiety, a tricostatin A moiety, a trapoxin B moiety, a valproic acid moiety, a mocetinostat moiety, an abexinostat moiety, an entinostat moiety, a resminostat moiety, a givinostat moiety, a quisinostat moiety, a pracinostat moiety, a sulforphane moiety, and pharmaceutically acceptable salts of any of the foregoing.

In some embodiments of any of the aforementioned embodiments, A² is selected from the group consisting of a moiety that inhibits histone deacetylase 1, a moiety that inhibits histone deacetylase 2, a moiety that inhibits histone deacetylase 3, a moiety that inhibits histone deacetylase 4, a moiety that inhibits histone deacetylase 5, a moiety that inhibits histone deacetylase 6, a moiety that inhibits histone deacetylase 7, a moiety that inhibits histone deacetylase 8, a moiety that inhibits histone deacetylase 9, a moiety that inhibits histone deacetylase 10, and a moiety that inhibits histone deacetylase 11. In some such embodiments, A² is a moiety that inhibits histone deacetylase 8. In some such embodiments, A² is a moiety that inhibits histone deacetylase 11.

In the aforementioned embodiments, the named moieties can have any suitable chemical form. In some embodiments of any of the aforementioned embodiments, the histone deacetylase inhibiting moieties are moieties where a hydrogen atom is absent from the named drug compound, or a pharmaceutically acceptable salt thereof. As a non-limiting example, such a “vorinostat moiety” would include the moiety of the following formula:

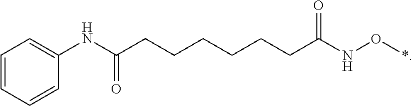

The structures below show various non-limiting examples of compounds that are included within the scope of compounds of formula (I), as defined above. Note that, at each occurrence, G is independently a hydrogen atom or —X²—X¹-A¹ (according to any of the aforementioned embodiments), wherein, for each compound, at least one G is not a hydrogen atom. In some embodiments, for each compound, exactly one G is —X²—X¹-A¹ (according to any of the aforementioned embodiments).

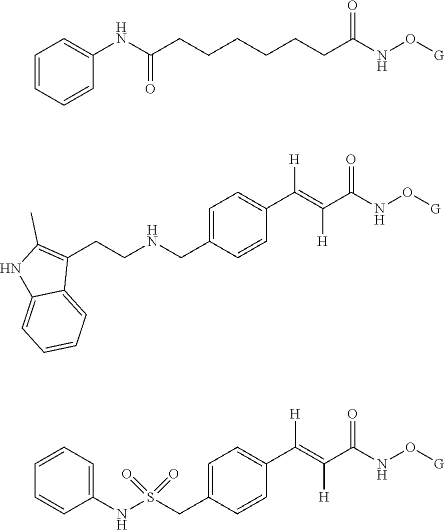

In the above structures, when G is it can be —X²—X¹-A¹ according to any of the previously recited embodiments, so long as those combinations result in stable chemical structures that would be suitable for pharmaceutical use. In some such embodiments, however, —X²—X¹-A¹ is —C(═O)—(CH₂)₁₀—CH₃, —C(═O)—(CH₂)₁₂—CH₃, —C(═O)—(CH₂)₁₄—CH₃, or —C(═O)—(CH₂)₁₆—CH₃. In some other such embodiments, —X²—X¹-A¹ is —C(═O)—(CH₂)₁₀—C(═O)—OH, —C(═O)—(CH₂)₁₂—C(═O)—OH, —C(═O)—(CH₂)₁₄—C(═O)—OH, or —C(═O)—(CH₂)₁₆—C(═O)—OH.

The selection of —X²—X¹-A¹ can depend on the nature of the connection to the drug moiety.

For example, in embodiments where the —X²—X¹-A¹ connects to an oxygen atom or an NH group on the drug moiety, as is the case for entries HAL HA2, and HA3 in Table 1, then —X²—X¹-A¹ is selected from the group consisting of: —C(═O)—(CH₂)ₙ₁—C(═O)—OH; —C(═O)—(CH₂)ₙ₁—C(═O)—OCH₃; —C(═O)—(CH₂)ₙ₁—CH₃; —C(═O)—(C₁₋₆ alkylene)-C(═O)—O—(CH₂)ₙ₂—C(═O)—OH; —C(═O)—(C₁₋₆ alkylene)-NH—C(═O)—(CH₂)ₙ₁—C(═O)—OH; —C(═O)—(C₁₋₆ alkylene)-C(═O)—O—[(CH₂)₂—O-]ₙ₃(CH₂)ₙ₂—C(═O)—OH; —C(═O)—O—(CH₂)ₙ₂—C(═O)—OH; and —C(═O)—NH—(CH₂)ₙ₂—C(═O)—OH; wherein n1 is an integer 12 to 24, n2 is an integer from 13 to 25, and n3 is an integer from 1 to 25. In some further such embodiments, —X²—X¹-A¹ is selected from the group consisting of: —C(═O)—(CH₂)ₙ₁—C(═O)—OH; —C(═O)—(CH₂)ₙ₁—C(═O)—OCH₃; —C(═O)—(C₁₋₆ alkylene)-C(═O)—O—(CH₂)ₙ₂—C(═O)—OH; —C(═O)—(C₁₋₆ alkylene)-NH—C(═O)—(CH₂)ₙ₁—C(═O)—OH; —C(═O)—(C₁₋₆ alkylene)-C(═O)—O—[(CH₂)₂—O—]ₙ₃(CH₂)ₙ₂—C(═O)—OH; —C(═O)—O—(CH₂)ₙ₂—C(═O)—OH; and —C(═O)—NH—(CH₂)ₙ₂—C(═O)—OH. In some further such embodiments, —X²—X¹-A¹ is selected from the group consisting of: —C(═O)—(CH₂)ₙ₁—C(═O)—OH; —C(═O)—O—(CH₂)ₙ₂—C(═O)—OH; and —C(═O)—NH—(CH₂)ₙ₂—C(═O)—OH. In some other embodiments, —X²—X¹-A¹ is —C(═O)—(C₁₋₆alkylene)-O—C(═O)—(CH₂)ₙ₁—C(═O)—OH, where n1 is an integer from 12 to 24. In some embodiments of any of the aforementioned embodiments, n1 is an integer from 14 to 22, or from 16 to 20. In some embodiments of any of the aforementioned embodiments, n2 is an integer from 15 to 23, or from 17 to 21. In some embodiments of any of the aforementioned embodiments, n3 is an integer from 1 to 15, or from 1 to 10, or from 1 to 6. In some such embodiments, —X²—X¹-A¹ is —C(═O)—(C₁₋₆ alkylene)-C(═O)—O—(CH₂)ₙ₃—OH, where n3 is an integer from 14 to 26, or an integer from 16 to 24, or an integer from 18 to 22.

In embodiments where the —X²—X¹-A¹ connects to an >N group on the drug moiety, then —X²—X¹-A¹ is selected from the group consisting of: —CH₂—O—C(═O)—(CH₂)ₙ₁—C(═O)—OH; —CH₂—O—C(═O)—(CH₂)ₙ₁—C(═O)—OCH₃; —CH₂—O—C(═O)—(CH₂)ₙ₁—CH₃; —CH₂—O—C(═O)—(C₁₋₆ alkylene)-C(═O)—O—(CH₂)ₙ₂—C(═O)—OH; —CH₂—O—C(═O)—(C₁₋₆ alkylene)-NH—C(═O)—(CH₂)ₙ₁—C(═O)—OH; —CH₂—O—C(═O)—(C₁₋₆ alkylene)-C(═O)—O—[(CH₂)₂—O—]ₙ₃(CH₂)ₙ₂—C(═O)—OH; —CH₂—O—C(═O)—O—(CH₂)ₙ₂—C(═O)—OH; and —CH₂—O—C(═O)—NH—(CH₂)ₙ₂—C(═O)—OH; wherein n1 is an integer 12 to 24, n2 is an integer from 13 to 25, and n3 is an integer from 1 to 25. In some further such embodiments, —X²—X¹-A¹ is selected from the group consisting of: —CH₂—O—C(═O)—(CH₂)ₙ₁—C(═O)—OH; —CH₂—O—C(═O)—(CH₂)ₙ₁—C(═O)—OCH₃; —CH₂—O—C(═O)—(C₁₋₆ alkylene)-C(═O)—O—(CH₂)ₙ₂—C(═O)—OH; —CH₂—O—C(═O)—(C₁₋₆ alkylene)-NH—C(═O)—(CH₂)ₙ₁—C(═O)—OH; —CH₂—O—C(═O)—(C₁₋₆ alkylene)-C(═O)—O—[(CH₂)₂—O—]ₙ₃(CH₂)ₙ₂—C(═O)—OH; —CH₂—O—C(═O)—O—(CH₂)ₙ₂—C(═O)—OH; and —C(═O)—NH—(CH₂)ₙ₂—C(═O)—OH. In some further such embodiments, —X²—X¹-A¹ is selected from the group consisting of: —CH₂—O—C(═O)—(CH₂)ₙ₁—C(═O)—OH; —CH₂—O—C(═O)—O—(CH₂)ₙ₂—C(═O)—OH; and —CH₂—O—C(═O)—NH—(CH₂)ₙ₂—C(═O)—OH. In some embodiments of any of the aforementioned embodiments, n1 is an integer from 14 to 22, or from 16 to 20. In some embodiments of any of the aforementioned embodiments, n2 is an integer from 15 to 23, or from 17 to 21. In some embodiments of any of the aforementioned embodiments, n3 is an integer from 1 to 15, or from 1 to 10, or from 1 to 6. In some such embodiments, —X²—X¹-A¹ is —CH₂—O—C(═O)—(C₁₋₆ alkylene)-C(═O)—O—(CH₂)ₙ₃—OH, where n3 is an integer from 14 to 26, or an integer from 16 to 24, or an integer from 18 to 22.

In embodiments where the —X²—X¹-A¹ connects to a —C(═O) group on the drug moiety, then —X²—X¹-A¹ is selected from the group consisting of: —O—(CH₂)ₙ₂—C(═O)—OH; —NH—(CH₂—C(═O)—OH; —NH—(C₁₋₆ alkylene)-O—C(═O)—(CH₂)ₙ₁—C(═O)—OH; —O—(C₁₋₆ alkylene)-O—C(═O)—(CH₂)ₙ₁—C(═O)—OH; —NH—(C₁₋₆ alkylene)-O—C(═O)—(CH₂)ₙ₁—C(═O)—OCH₃; —O—(C₁₋₆ alkylene)-O—C(═O)—(CH₂)ₙ₁—C(═O)—OCH₃; —NH—(C₁₋₆alkylene)-O—C(═O)—(CH₂)ₙ₁—CH₃; —O—(C₁₋₆ alkylene)-O—C(═O)—(CH₂)ₙ₁—CH₃; —NH—(C₁₋₆ alkylene)-C(═O)—O—[(CH₂)₂—O—]ₙ₃(CH₂)ₙ₂—C(═O)—OH; and —O—(C₁₋₆ alkylene)-C(═O)—O—[(CH₂)₂—O—]ₙ₃(CH₂)ₙ₂—C(═O)—OH; wherein n1 is an integer 12 to 24, n2 is an integer from 13 to 25, and n3 is an integer from 1 to 25. In some further such embodiments, —X²—X¹-A¹ is selected from the group consisting of: —O—(CH₂)ₙ₂—C(═O)—OH; —NH—(CH₂)ₙ₂—C(═O)—OH; —NH—(C₁₋₆ alkylene)-O—C(═O)—(CH₂)ₙ₁—C(═O)—OH; —O—(C₁₋₆ alkylene)-O—C(═O)—(CH₂)ₙ₁—C(═O)—OH; —NH—(C₁₋₆alkylene)-O—C(═O)—(CH₂)ₙ₁—C(═O)—OCH₃; and —O—(C₁₋₆ alkylene)-O—C(═O)—(CH₂)ₙ₁—C(═O)—OCH₃. In some further such embodiments, —X²—X¹-A¹ is selected from the group consisting of: —O—(CH₂)ₙ₂—C(═O)—OH; —NH—(CH₂)ₙ₂—C(═O)—OH; —NH—(C₁₋₆ alkylene)-O—C(═O)—(CH₂)ₙ₁—C(═O)—OH; and —O—(C₁₋₆alkylene)-O—C(═O)—(CH₂)ₙ₁—C(═O)—OH. In some embodiments of any of the aforementioned embodiments, n1 is an integer from 14 to 22, or from 16 to 20. In some embodiments of any of the aforementioned embodiments, n2 is an integer from 15 to 23, or from 17 to 21. In some embodiments of any of the aforementioned embodiments, n3 is an integer from 1 to 15, or from 1 to 10, or from 1 to 6. In some such embodiments, —X²—X¹-A¹ is —O—(CH₂)ₙ₃—OH, where n3 is an integer from 14 to 26, or an integer from 16 to 24, or an integer from 18 to 22.

In embodiments where the —X²—X¹-A¹ connects to a C═* group on the drug moiety, then —X²—X¹-A¹ is selected from the group consisting of: ═N—O—(CH₂)ₙ₂—C(═O)—OH; ═N—NH—(CH₂)ₙ₂—C(═O)—OH; ═N—O—(CH₂)ₙ₂—C(═O)—OCH₃; ═N—NH—(CH₂)ₙ₂—C(═O)—OCH₃; ═N—O—(CH₂)ₙ₂—CH₃; ═N—NH—(CH₂)ₙ₂—CH₃; N—O—[(CH₂)₂—O—]ₙ₃(CH₂)ₙ₂—C(═O)—OH; and ═N—NH—[(CH₂)₂—O—]ₙ₃(CH₂)ₙ₂—C(═O)—OH; n2 is an integer from 13 to 25, and n3 is an integer from 1 to 25. In some further such embodiments, —X²—X¹-A¹ is selected from the group consisting of: ═N—O—(CH₂)ₙ₂—C(═O)—OH; ═N—NH—(CH₂)ₙ₂—C(═O)—OH; ═N—O—(CH₂)ₙ₂—C(═O)—OCH₃; and ═N—NH—(CH₂)ₙ₂—C(═O)—OCH₃. In some further such embodiments, —X²—X¹-A¹ is selected from the group consisting of: ═N—O—(CH₂)ₙ₂—C(═O)—OH and ═N—NH—(CH₂)ₙ₂—C(═O)—OH. In some embodiments of any of the aforementioned embodiments, n2 is an integer from 15 to 23, or from 17 to 21. In some embodiments of any of the aforementioned embodiments, n3 is an integer from 1 to 15, or from 1 to 10, or from 1 to 6. In some such embodiments, —X²—X¹-A¹ is selected from the group consisting of: ═N—O—(CH₂)ₙ₃—OH and ═N—NH—(CH₂)ₙ₃—OH, where n3 is an integer from 14 to 26, or an integer from 16 to 24, or an integer from 18 to 22.

The compounds described in any of the above embodiments can also exist as pharmaceutically acceptable salts. The term “pharmaceutically acceptable salts” refers to salts of the compounds which are not biologically or otherwise undesirable and are generally prepared by reacting the free base with a suitable organic or inorganic acid or by reacting the acid with a suitable organic or inorganic base. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium, and valerate. When an acidic substituent is present, such as —COOH, there can be formed the ammonium, morpholinium, sodium, potassium, barium, calcium salt, and the like, for use as the dosage form. When a basic group is present, such as amino or a basic heteroaryl radical, such as pyridyl, there can be formed an acidic salt, such as hydrochloride, hydrobromide, phosphate, sulfate, trifluoroacetate, trichloroacetate, acetate, oxalate, maleate, pyruvate, malonate, succinate, citrate, tartarate, fumarate, mandelate, benzoate, cinnamate, methanesulfonate, ethanesulfonate, picrate, and the like.

The compounds above can be made by standard organic synthetic methods, such as those illustrated in: Wuts et al., Greene's Protective Groups in Organic Synthesis (4th ed., 2006); Larock, Comprehensive Organic Transformations (2nd ed., 1999); and Smith et al., March's Advanced Organic Chemistry: Reactions, Mechanisms, and Structure (6th ed., 2007). Specific non-limiting examples are shown below in the Examples.

The compounds of the foregoing embodiments, including their pharmaceutically acceptable salts, are useful as histone deacetylase inhibiting compounds or prodrugs thereof, and are therefore useful as compounds for the treatment of cancer.

Table 3 (below) shows various examples of compounds that are contemplated by the present disclosure. Table 3 refers to various combinations of an A²- moiety with a —X²—X¹-A¹, which together form compounds of the present disclosure. Table 1 shows illustrative example moieties for the A²- moiety, wherein A² can be the moiety shown or can also be a pharmaceutically acceptable salt thereof. Table 2 shows illustrative example moieties for —X²—X¹-A¹. Table 3 shows non-limiting illustrative combinations of the moieties from Tables 1 and 2, which can come together to form compounds of the present disclosure. The compounds disclosed in Table 3 can be made by methods analogous to those illustrated in the Examples, and by common synthetic methods known to those of ordinary skill in the art. Suitable methods of making such compounds are illustrated in: Wuts et al., Greene's Protective Groups in Organic Synthesis (4th ed., 2006); Larock, Comprehensive Organic Transformations (2nd ed., 1999); and Smith et al., March's Advanced Organic Chemistry: Reactions, Mechanisms, and Structure (6th ed., 2007).

TABLE 1
A²-Moieties

| | |
|---|---|
| HA1 | 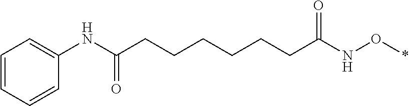 [SMILES: *ONC(=O)CCCCCCC(=O)Nc1ccccc1] |
| | a vorinostat moiety |
| HA2 | 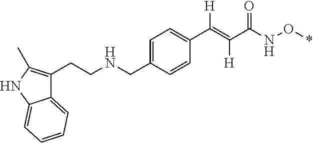 [SMILES: *ONC(=O)/C=C/c1ccc(CNCCc2c(C)[nH]c3ccccc23)cc1] |
| | a panobinostat moiety |
| HA3 | 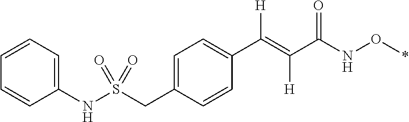 [SMILES: *ONC(=O)/C=C/c1ccc(CS(=O)(=O)Nc2ccccc2)cc1] |
| | a belinostat moiety |

TABLE 2

| | —X²—X¹—A¹ Moieties |
|---|---|
| HB1 | —C(═O)—(CH₂)₁₄—C(═O)—OH |
| HB2 | —C(═O)—(CH₂)₁₆—C(═O)—OH |
| HB3 | —C(═O)—(CH₂)₁₈—C(═O)—OH |
| HB4 | —C(═O)—(CH₂)₇—CH═CH—(CH₂)₇—C(═O)—OH |
| HB5 | —C(═O)—(CH₂)₁₄—C(═O)—O—CH₃ |
| HB6 | —C(═O)—(CH₂)₁₆—C(═O)—O—CH₃ |
| HB7 | —C(═O)—(CH₂)₁₈—C(═O)—O—CH₃ |
| HB8 | —C(═O)—(CH₂)₇—CH═CH—(CH₂)₇—C(═O)—O—CH₃ |
| HB9 | —C(═O)—(CH₂)₁₄—CH₃ |
| HB10 | —C(═O)—(CH₂)₁₆—CH₃ |
| HB11 | —C(═O)—(CH₂)₁₈—CH₃ |
| HB12 | —C(═O)—(CH₂)₇—CH═CH—(CH₂)₇—CH₃ |
| HB13 | —C(═O)—(CH₂)₂—C(═O)—O—(CH₂)₁₅—C(═O)—OH |
| HB14 | —C(═O)—(CH₂)₂—C(═O)—O—(CH₂)₁₇—C(═O)—OH |
| HB15 | —C(═O)—(CH₂)₂—C(═O)—O—(CH₂)₁₉—C(═O)—OH |
| HB16 | —C(═O)—(CH₂)₂—C(═O)—O—(CH₂)₈—CH═CH—(CH₂)₇—C(═O)—OH |
| HB17 | —C(═O)—CH₂—NH—C(═O)—(CH₂)₁₄—C(═O)—OH |
| HB18 | —C(═O)—CH₂—NH—C(═O)—(CH₂)₁₆—C(═O)—OH |
| HB19 | —C(═O)—CH₂—NH—C(═O)—(CH₂)₁₈—C(═O)—OH |
| HB20 | —C(═O)—CH₂—NH—C(═O)—(CH₂)₇—CH═CH—(CH₂)₇—C(═O)—OH |
| HB21 | —C(═O)—(CH₂)₂—C(═O)—O—[(CH₂)₂—O—]₆C(═O)—(CH₂)₁₄—C(═O)—OH |
| HB22 | —C(═O)—(CH₂)₂—C(═O)—O—[(CH₂)₂—O—]₆C(═O)—(CH₂)₁₆—C(═O)—OH |
| HB23 | —C(═O)—(CH₂)₂—C(═O)—O—[(CH₂)₂—O—]₆C(═O)—(CH₂)₁₈—C(═O)—OH |
| HB24 | —C(═O)—(CH₂)₂—C(═O)—O—[(CH₂)₂—O—]₆C(═O)—(CH₂)₇—CH═CH—(CH₂)₇—C(═O)—OH |
| HB25 | —C(═O)—O—(CH₂)₁₅—C(═O)—OH |
| HB26 | —C(═O)—O—(CH₂)₁₇—C(═O)—OH |
| HB27 | —C(═O)—O—(CH₂)₁₉—C(═O)—OH |
| HB28 | —C(═O)—O—(CH₂)₈—CH═CH—(CH₂)₇—C(═O)—OH |
| HB29 | —C(═O)—NH—(CH₂)₁₅—C(═O)—OH |
| HB30 | —C(═O)—NH—(CH₂)₁₇—C(═O)—OH |
| HB31 | —C(═O)—NH—(CH₂)₁₉—C(═O)—OH |
| HB32 | —C(═O)—NH—(CH₂)₈—CH═CH—(CH₂)₇—C(═O)—OH |
| HB33 | —O—(CH₂)₁₅—C(═O)—OH |
| HB34 | —O—(CH₂)₁₇—C(═O)—OH |
| HB35 | —O—(CH₂)₁₉—C(═O)—OH |
| HB36 | —O—(CH₂)₈—CH═CH—(CH₂)₇—C(═O)—OH |
| HB37 | —NH—(CH₂)₂—O—C(═O)—(CH₂)₁₄—C(═O)—OH |
| HB38 | —NH—(CH₂)₂—O—C(═O)—(CH₂)₁₆—C(═O)—OH |
| HB39 | —NH—(CH₂)₂—O—C(═O)—(CH₂)₁₈—C(═O)—OH |
| HB40 | —NH—(CH₂)₂—O—C(═O)—(CH₂)₇—CH═CH—(CH₂)₇—C(═O)—OH |
| HB41 | —O—(CH₂)₂—O—C(═O)—(CH₂)₁₄—C(═O)—OH |
| HB42 | —O—(CH₂)₂—O—C(═O)—(CH₂)₁₆—C(═O)—OH |
| HB43 | —O—(CH₂)₂—O—C(═O)—(CH₂)₁₈—C(═O)—OH |
| HB44 | —O—(CH₂)₂—O—C(═O)—(CH₂)₇—CH═CH—(CH₂)₇—C(═O)—OH |
| HB45 | —NH—CH₂—C(═O)—O—[(CH₂)₂—O—]₆C(═O)—(CH₂)₁₄—C(═O)—OH |
| HB46 | —NH—CH₂—C(═O)—O—[(CH₂)₂—O—]₆C(═O)—(CH₂)₁₆—C(═O)—OH |
| HB47 | —NH—CH₂—C(═O)—O—[(CH₂)₂—O—]₆C(═O)—(CH₂)₁₈—C(═O)—OH |
| HB48 | —NH—CH₂—C(═O)—O—[(CH₂)₂—O—]₆C(═O)—(CH₂)₇—CH═CH—(CH₂)₇—C(═O)—OH |
| HB49 | —NH—(CH₂)₂—O—C(═O)—(CH₂)₁₄—C(═O)—O—CH₃ |
| HB50 | —NH—(CH₂)₂—O—C(═O)—(CH₂)₁₆—C(═O)—O—CH₃ |
| HB51 | —NH—(CH₂)₂—O—C(═O)—(CH₂)₁₈—C(═O)—O—CH₃ |
| HB52 | —NH—(CH₂)₂—O—C(═O)—(CH₂)₇—CH═CH—(CH₂)₇—C(═O)—O—CH₃ |
| HB53 | —CH₂—O—C(═O)—(CH₂)₁₄—C(═O)—OH |
| HB54 | —CH₂—O—C(═O)—(CH₂)₁₆—C(═O)—OH |
| HB55 | —CH₂—O—C(═O)—(CH₂)₁₈—C(═O)—OH |
| HB56 | —CH₂—O—C(═O)—(CH₂)₇—CH═CH—(CH₂)₇—C(═O)—OH |
| HB57 | —CH₂—O—C(═O)—(CH₂)₁₄—C(═O)—O—CH₃ |
| HB58 | —CH₂—O—C(═O)—(CH₂)₁₆—C(═O)—O—CH₃ |
| HB59 | —CH₂—O—C(═O)—(CH₂)₁₈—C(═O)—O—CH₃ |
| HB60 | —CH₂—O—C(═O)—(CH₂)₇—CH═CH—(CH₂)₇—C(═O)—O—CH₃ |
| HB61 | —CH₂—O—C(═O)—(CH₂)₁₄—CH₃ |
| HB62 | —CH₂—O—C(═O)—(CH₂)₁₆—CH₃ |
| HB63 | —CH₂—O—C(═O)—(CH₂)₁₈—CH₃ |
| HB64 | —CH₂—O—C(═O)—(CH₂)₇—CH═CH—(CH₂)₇—CH₃ |
| HB65 | —CH₂—O—C(═O)—CH₂—NH—C(═O)—(CH₂)₁₄—C(═O)—OH |
| HB66 | —CH₂—O—C(═O)—CH₂—NH—C(═O)—(CH₂)₁₆—C(═O)—OH |
| HB67 | —CH₂—O—C(═O)—CH₂—NH—C(═O)—(CH₂)₁₈—C(═O)—OH |
| HB68 | —CH₂—O—C(═O)—CH₂—NH—C(═O)—(CH₂)₇—CH═CH—(CH₂)₇—C(═O)—OH |
| HB69 | —CH₂—O—C(═O)—(CH₂)₂—C(═O)—O—[(CH₂)₂—O—]₆C(═O)—(CH₂)₁₄—C(═O)—OH |
| HB70 | —CH₂—O—C(═O)—(CH₂)₂—C(═O)—O—[(CH₂)₂—O—]₆C(═O)—(CH₂)₁₆—C(═O)—OH |
| HB71 | —CH₂—O—C(═O)—(CH₂)₂—C(═O)—O—[(CH₂)₂—O—]₆C(═O)—(CH₂)₁₈—C(═O)—OH |
| HB72 | —CH₂—O—C(═O)—(CH₂)₂—C(═O)—O—][(CH₂)₂—O—]₆C(═O)—(CH₂)₇—CH═CH—(CH₂)₇ |
| | —C(═O)—OH |
| HB73 | —CH₂—O—C(═O)—O—(CH₂)₁₅—C(═O)—OH |
| HB74 | —CH₂—O—C(═O)—O—(CH₂)₁₇—C(═O)—OH |
| HB75 | —CH₂—O—C(═O)—O—(CH₂)₁₉—C(═O)—OH |
| HB76 | —CH₂—O—C(═O)—O—(CH₂)₈—CH═CH—(CH₂)₇—C(═O)—OH |
| HB77 | —CH₂—O—C(═O)—NH—(CH₂)₁₅—C(═O)—OH |
| HB78 | —CH₂—O—C(═O)—NH—(CH₂)₁₇—C(═O)—OH |
| HB79 | —CH₂—O—C(═O)—NH—(CH₂)₁₉—C(═O)—OH |
| HB80 | —CH₂—O—C(═O)—NH—(CH₂)₈—CH═CH—(CH₂)₇—C(═O)—OH |
| HB81 | ═N—O—(CH₂)₁₅—C(═O)—OH |
| HB82 | ═N—O—(CH₂)₁₇—C(═O)—OH |
| HB83 | ═N—O—(CH₂)₁₉—C(═O)—OH |
| HB84 | ═N—O—(CH₂)₈—CH═CH—(CH₂)₇—C(═O)—OH |
| HB85 | ═N—NH—(CH₂)₁₅—C(═O)—OH |
| HB86 | ═N—NH—(CH₂)₁₇—C(═O)—OH |
| HB87 | ═N—NH—(CH₂)₁₉—C(═O)—OH |
| HB88 | ═N—NH—(CH₂)₈—CH═CH—(CH₂)₇—C(═O)—OH |
| HB89 | ═N—O—[(CH₂)—O—]₆(CH₂)₁₅—C(═O)—OH |
| HB90 | ═N—O—[(CH₂)—O—]₆(CH₂)₁₇—C(═O)—OH |
| HB91 | ═N—O—[(CH₂)₂—O—]₆(CH₂)₁₉—C(═O)—OH |
| HB92 | ═N—O—[(CH₂)₂—O—]₆(CH₂)₈—CH═CH—(CH₂)₇—C(═O)—OH |
| HB93 | —C(═O)—CH₂—O—C(═O)—(CH₂)₁₄—C(═O)—OH |
| HB94 | —C(═O)—CH₂—O—C(═O)—(CH₂)₁₆—C(═O)—OH |
| HB95 | —C(═O)—CH₂—O—C(═O)—(CH₂)₁₈—C(═O)—OH |
| HB96 | —C(═O)—CH₂—O—C(═O)—(CH₂)₇—CH═CH—(CH₂)₇—C(═O)—OH |
| HB97 | —C(═O)—CH(CH₃)—O—C(═O)—(CH₂)₁₄—C(═O)—OH |
| HB98 | —C(═O)—CH(CH₃)—O—C(═O)—(CH₂)₁₆—C(═O)—OH |
| HB99 | —C(═O)—CH(CH₃)—O—C(═O)—(CH₂)₁₈—C(═O)—OH |
| HB100 | —C(═O)—CH(CH₃)—O—C(═O)—(CH₂)₇—CH═CH—(CH₂)₇—C(═O)—OH |
| HB101 | —C(═O)—(CH₂)₅—O—C(═O)—(CH₂)₁₄—C(═O)—OH |
| HB102 | —C(═O)—(CH₂)₅—O—C(═O)—(CH₂)₁₆—C(═O)—OH |
| HB103 | —C(═O)—(CH₂)₅—O—C(═O)—(CH₂)₁₈—C(═O)—OH |
| HB104 | —C(═O)—(CH₂)₅—O—C(═O)—(CH₂)₇—CH═CH—(CH₂)₇—C(═O)—OH |
| HB105 | —C(═O)—NH—(CH₂)₁₄—C(═O)—OH |
| HB106 | —C(═O)—NH—(CH₂)₁₆—C(═O)—OH |
| HB107 | —C(═O)—NH—(CH₂)₁₈—C(═O)—OH |
| HB108 | —C(═O)—NH—(CH₂)₇—CH═CH—(CH₂)₇—C(═O)—OH |

TABLE 3

| Compound | A²— | —X²—X¹—A¹ |
|---|---|---|
| No. | Moiety | Moiety |
| 1-48 | HA1 | HB1, HB2, HB3, HB4, HB5, HB6, HB7, HB8, HB9, |
| | | HB10, HB11, HB12, HB13, HB14, HB15, HB16, |
| | | HB17, HB18, HB19, HB20, HB21, HB22, HB23, |
| | | HB24, HB25, HB26, HB27, HB28, HB29, HB30, |
| | | HB31, HB32, HB93, HB94, HB95, HB96, HB97, |
| | | HB98, HB99, HB100, HB101, HB102, HB103, |
| | | HB104, HB105, HB106, HB107, HB108, |
| | | respectively |
| 49-96 | HA2 | HB1, HB2, HB3, HB4, HB5, HB6, HB7, HB8, HB9, |
| | | HB10, HB11, HB12, HB13, HB14, HB15, HB16, |
| | | HB17, HB18, HB19, HB20, HB21, HB22, HB23, |
| | | HB24, HB25, HB26, HB27, HB28, HB29, HB30, |
| | | HB31, HB32, HB93, HB94, HB95, HB96, HB97, |
| | | HB98, HB99, HB100, HB101, HB102, HB103, |
| | | HB104, HB105, HB106, HB107, HB108, |
| | | respectively |
| 97-144 | HA3 | HB1, HB2, HB3, HB4, HB5, HB6, HB7, HB8, HB9, |
| | | HB10, HB11, HB12, HB13, HB14, HB15, HB16, |
| | | HB17, HB18, HB19, HB20, HB21, HB22, HB23, |
| | | HB24, HB25, HB26, HB27, HB28, HB29, HB30, |
| | | HB31, HB32, HB93, HB94, HB95, HB96, HB97, |
| | | HB98, HB99, HB100, HB101, HB102, HB103, |
| | | HB104, HB105, HB106, HB107, HB108, |
| | | respectively |

Pharmaceutical Compositions

In certain aspects, the compounds of any of the preceding embodiments may be formulated into pharmaceutical compositions in any suitable manner. In general, as compounds for the treatment of cancer, such pharmaceutical formulations are aqueous formulations suitable for parenteral administration, such as intravenous or intra-arterial administration.

In at least one aspect, the disclosure provides pharmaceutical compositions that include one or more compounds of formula (I) (according to any of the foregoing embodiments) and a protein. In some embodiments, the protein is an albumin or an albumin mimetic. In some such embodiments, the protein is human serum albumin (HSA) or a mimetic thereof, i.e., a protein whose sequence is at least 50% equivalent to that of HSA, or at least 60% equivalent to that of HSA, or at least 70% equivalent to that of HSA, or at least 80% equivalent to that of HSA, or at least 90% equivalent to that of HSA, or at least 95% equivalent to that of HSA, at least 97% equivalent to that of HSA, at least 99% equivalent to that of HSA. In some embodiments, the protein is human serum albumin.

In certain embodiments of any of the foregoing embodiments, the pharmaceutical composition also includes a carrier, such as a liquid carrier. In some embodiments, the carrier includes water. For example, in some such embodiments, water makes up at least 50% by volume, or at least 60% by volume, or at least 70% by volume, or at least 80% by volume, or at least 90% by volume, based on the total volume of liquid materials in the pharmaceutical composition. The carrier can also include other liquid ingredients, such as liquid ingredients commonly included in aqueous pharmaceutical formulations for parenteral administration.

In certain embodiments having an aqueous carrier, the compounds of formula (I) bind non-covalently to the protein in the pharmaceutical formulation. In some embodiments, the compound of formula (I) and the protein (e.g., human serum albumin) are non-covalently associated with each other with a binding constant (K_{b}) of at least 10² M⁻¹, or at least 10³ M⁻¹, or at least 10⁴ M⁻¹, or at least 10⁵ M⁻¹ at 25° C. in the aqueous composition.

In some embodiments having an aqueous carrier, the compound of formula (I) and the protein are solvated by the carrier. In some such embodiments, at least 90% by weight, or at least 95% by weight, or at least 97% by weight, or at least 98% by weight, or at least 99% by weight of the compounds of formula (I) in the composition are bound non-covalently to the protein with a binding constant (K_{b}) of at least 10² M⁻¹, or at least 10³ M⁻¹, or at least 10⁴ M⁻¹, or at least 10⁵ M⁻¹ at 25° C. in the aqueous composition. In some further such embodiments, the composition is substantially free of agglomerates or nanoparticles. For example, in some embodiments of any of the aforementioned embodiments, no more than 5% by weight, or no more than 4% by weight, or no more than 3% by weight, or no more than 2% by weight, or no more than 1% by weight of the protein-compound (i.e., non-covalently bound conjugates between the protein and one or more compounds of formula (I)) in the aqueous composition have a radius greater than 7 nm, or a radius greater than 5 nm, or a radius greater than 4 nm, as measured by dynamic light scattering.

The compound of formula (I) can have any suitable molar ratio to the protein in the formulation. For example, in some embodiments of any of the foregoing embodiments, the molar ratio of the compound of formula (I) to the protein ranges from 1:10 to 20:1, or from 1:5 to 15:1, or from 1:2 to 10:1. In some embodiments of any of the foregoing embodiments, the molar ratio of the compound of formula (I) to the protein is about 1:1, or is about 2:1, or is about 3:1, or is about 4:1, or is about 5:1, or is about 6:1, or is about 7:1, wherein the term “about,” in this instance means±0.5:1, such that “about 5:1” refers to a range from 4.5:1 to 5.5:1.

In at least one aspect, the disclosure provides pharmaceutical compositions that include: a compound, which comprises a histone deacetylase inhibiting moiety and a protein binding moiety; a protein, wherein the protein is an albumin or an albumin mimetic; and a carrier, which comprises water.

In some embodiments, the protein is human serum albumin (HSA) or a mimetic thereof, i.e., a protein whose sequence is at least 50% equivalent to that of HSA, or at least 60% equivalent to that of HSA, or at least 70% equivalent to that of HSA, or at least 80% equivalent to that of HSA, or at least 90% equivalent to that of HSA, or at least 95% equivalent to that of HSA, at least 97% equivalent to that of HSA, at least 99% equivalent to that of HSA. In some embodiments, the protein is human serum albumin.

As noted above, in some embodiments, the carrier includes water. For example, in some such embodiments, water makes up at least 50% by volume, or at least 60% by volume, or at least 70% by volume, or at least 80% by volume, or at least 90% by volume, based on the total volume of liquid materials in the pharmaceutical composition. The carrier can also include other liquid ingredients, such as liquid ingredients commonly included in aqueous pharmaceutical formulations for parenteral administration.

In certain embodiments, the compounds bind non-covalently to the protein in the pharmaceutical formulation. In some embodiments, the compound and the protein (e.g., human serum albumin) are non-covalently associated with each other with a binding constant (K_{b}) of at least 10² M⁻¹, or at least 10³ M⁻¹, or at least 10⁴ M⁻¹, or at least 10⁵ M⁻¹ at 25° C. in the aqueous composition.

In some embodiments having an aqueous carrier, the compound and the protein are solvated by the carrier. In some such embodiments, at least 90% by weight, or at least 95% by weight, or at least 97% by weight, or at least 98% by weight, or at least 99% by weight of the compounds of formula (I) in the composition are bound non-covalently to the protein with a binding constant (K_{b}) of at least 10² M⁻¹, or at least 10³ M⁻¹, or at least 10⁴ M⁻¹, or at least 10⁵ M⁻¹ at 25° C. in the aqueous composition. In some further such embodiments, the composition is substantially free of agglomerates or nanoparticles. For example, in some embodiments of any of the aforementioned embodiments, no more than 5% by weight, or no more than 4% by weight, or no more than 3% by weight, or no more than 2% by weight, or no more than 1% by weight of the protein-compound (i.e., non-covalently bound conjugates between the protein and one or more compounds of formula (I)) in the aqueous composition have a radius greater than 7 nm, or a radius greater than 5 nm, or a radius greater than 4 nm, as measured by dynamic light scattering.

The compound of formula (I) can have any suitable molar ratio to the protein in the formulation. For example, in some embodiments of any of the foregoing embodiments, the molar ratio of the compound of formula (I) to the protein ranges from 1:10 to 20:1, or from 1:5 to 15:1, or from 1:2 to 10:1. In some embodiments of any of the foregoing embodiments, the molar ratio of the compound of formula (I) to the protein is about 1:1, or is about 2:1, or is about 3:1, or is about 4:1, or is about 5:1, or is about 6:1, or is about 7:1, wherein the term “about,” in this instance means±0.5:1, such that “about 5:1” refers to a range from 4.5:1 to 5.5:1.

The pharmaceutical compositions of any of the foregoing aspects and embodiments can also include certain additional ingredients, such as those commonly employed in pharmaceutical compositions for parenteral administration.

### Methods and Uses

The compounds or compositions of any of the foregoing embodiments are useful in the treatment of cancer and related disorders. Therefore, these compounds and compositions can be used for administration to a subject who has or has had a cancerous tumor.

Thus, in certain aspects, the disclosure provides methods of treating cancer, including administering to a subject a compound or composition of any of the foregoing aspects and embodiments. In some embodiments, the subject is a human. In some embodiments, the subject is a subject in need of such treatment, e.g., a human in need of such treatment.

In some aspects, the disclosure provides methods of inducing apoptosis in a cancer cell, including contacting the cancer cell with a compound or composition of any of the foregoing aspects and embodiments.

In some aspects, the disclosure provides methods of inhibiting proliferation of a cancerous tumor, including contacting the cancerous tumor with a compound or composition of any of the foregoing aspects and embodiments.

In some aspects, the disclosure provides uses of a compound or composition of any of the foregoing aspects and embodiments as a medicament.

In some aspects, the disclosure provides uses of a compound or composition of any of the foregoing aspects and embodiments for treating cancer.

In some aspects, the disclosure provides uses of a compound of any of the foregoing aspects and embodiments in the manufacture of a medicament.

In some aspects, the disclosure provides uses of a compound of any of the foregoing aspects and embodiments in the manufacture of a medicament for treating cancer.

### Combination Therapies

The compounds or compositions of any of the foregoing embodiments are useful when used in conjunction with immunotherapy agents, such as checkpoint inhibitors, toll like receptor modulators, and various antibodies, including, but not limited to, alemtuzumab, atezolizumab, ipilimumab, ofatumumab, nivolumab, pembrolizumab, and rituximab.

### EXAMPLES

The following examples show certain illustrative embodiments of the compounds, compositions, and methods disclosed herein. These examples are not to be taken as limiting in any way. Nor should the examples be taken as expressing any preferred embodiments, or as indicating any direction for further research.

The examples may use abbreviations for certain common chemicals. The following abbreviations refer to the compounds indicated.
DMF=Dimethylformamide
DBTL=Dibutyltin dilaurate
DCM=Dichloromethane
NMR=Nuclear magnetic resonance
HPLC=High-performance liquid chromatography
RP-HLPC=Reverse-phase high-performance liquid chromatography
LRMS=Liquid chromatography/low-resolution mass spectrometry
HRMS=Liquid chromatography/high-resolution mass spectrometry
Tips=Triisopropylsilyl
DMAP=4-(Dimethylamino)pyridine
EDC=1-Ethyl-3-(3-dimethylaminopropyl)carbodiimide
THF=Tetrahydrofuran
Dipea=N,N-diisopropylethylamine
HATU=1-[Bis(dimethylamino)methylene]-1H-1,2,3-triazolo-[4,5-b]pyridinium 3-oxide hexafluorophosphate
DCC=N,N′-dicyclohexylcarbodiimide
HSA=Human serum albumin

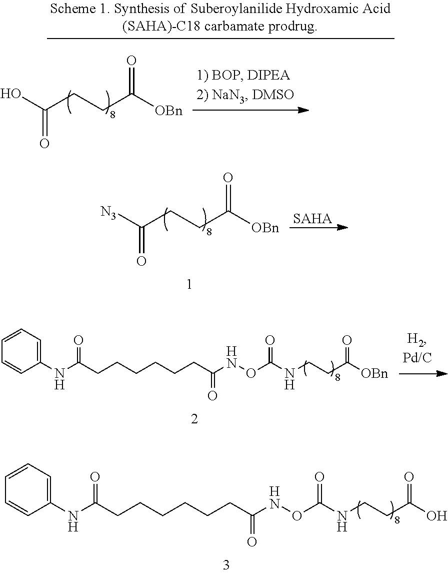

### Example 1—General Synthetic Procedures from Scheme 1

Synthesis of (1).

To a solution of mono-benzyl protected octadecanedioic acid (1.0 mmol) in THF (10 mL), is added BOP (1.1 mmol) at 0° C. The reaction mixture is stirred for 30 min. To this is added NaN₃ (1.5 mmol) in DMSO (5 mL) and stirring continued for an additional 30 min. The reaction is concentrated under reduced pressure, diluted in DCM (30 mL) and washed with aqueous 10% sodium bicarbonate solution, brine, and dried over Na₂SO₄. The solvent was concentrated in vacuo (without heating) to obtain the acyl azide 1.

Synthesis of (2).

To a solution of 1 (0.1 mmol) in dry toluene (10 mL) is added SAHA (0.09 mmol) and DBTL (0.001 mmol). The reaction is heated to reflux at 110° C. for 8 h. Upon completion, the reaction is cooled to rt and concentrated under reduced pressure. The crude residue is purified by flash chromatography (2% MeOH:DCM) to yield 2.

Synthesis of (3).

General benzyl ester deprotection conditions via hydrogenolysis are employed. To a solution of 2 (0.05 mmol) in MeOH (20 mL) is added 10% Pd/C (25 mg) in a suitable pressure vessel. The mixture is pressurized with hydrogen (5 bar) and stirred for 3 h. The catalyst is removed via vacuum filtration and the filtrate concentrated under reduced pressure. The crude residue is purified by flash chromatography (5% MeOH:DCM) to yield 3.

1. A compound of formula (I)
A¹-X¹—X²-A² (I)
wherein:
A¹ is an organic group; or A¹ is a hydrophilic group or a hydrogen atom;
A² is a histone deacetylase inhibiting moiety;
X¹ is a hydrophobic group; and
X² is a direct bond, an organic group, —O—, —S—, —S(═O)—, —S(═O)₂—, —S—S—, —N═, ═N—, —N(H)—, —N═N—N(H)—, —N(H)—N═N—, —N(OH)—, or —N(═O)—.

2. The compound of claim 1, wherein A¹ is a carboxylic acid group, a carboxylate anion, or a carboxylate ester.

3. The compound of claim 2, wherein A¹ is a carboxylic acid group.

4. The compound of any one of claims 1 to 3, wherein the histone deacetylase inhibiting moiety has a molecular weight of no more than 1600 Da, no more than 1500 Da, or no more than 1400 Da, or no more than 1300 Da, or no more than 1200 Da, or no more than 1100 Da, or no more than 1000 Da.

5. The compound of any one of claims 1 to 4, wherein the histone deacetylase inhibiting moiety is an organic moiety.

6. The compound of any one of claims 1 to 5, wherein the histone deacetylase inhibiting moiety is a vorinostat moiety, a romidepsin moiety, a chidamide moiety, a panobinostat moiety, a belinostat moiety, a tricostatin A moiety, a trapoxin B moiety, a valproic acid moiety, a mocetinostat moiety, an abexinostat moiety, an entinostat moiety, a resminostat moiety, a givinostat moiety, a quisinostat moiety, a pracinostat moiety, a sulforphane moiety, or a pharmaceutically acceptable salt of any of the foregoing.

7. The compound of claim 6, wherein the histone deacetylase inhibiting moiety is selected from the group consisting of a vorinostat moiety, a panobinostat moiety, a belinostat moiety, and pharmaceutically acceptable salts of any of the foregoing.

8. The compound of claim 7, wherein the histone deacetylase inhibiting moiety is a vorinostat moiety.

9. The compound of claim 8, wherein the vorinostat moiety is a moiety of the formula:

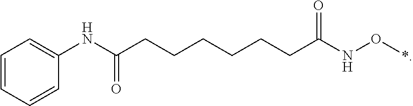

10. The compound of claim 8, wherein the histone deacetylase inhibiting moiety is a panobinostat moiety, and wherein the panobinostat moiety is a moiety of the formula:

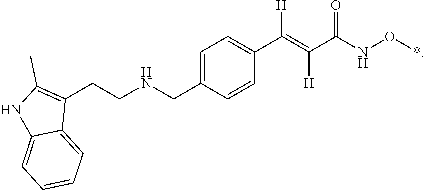

11. The compound of any one of claims 1 to 10, wherein X¹ is C₁₂₋₂₂ hydrocarbylene, which is optionally substituted.

12. The compound of claim 11, wherein X¹ is C₁₂₋₂₂ alkylene group.

13. The compound of claim 12, wherein X¹ is —(CH₂)₁₂—, —(CH₂)₁₄—, —(CH₂)₁₆—, —(CH₂)₁₈—, —(CH₂)₂₀—, or —(CH₂)₂₂—.

14. The compound of claim 13, wherein X¹ is —(CH₂)₁₆—.

15. The compound of claim 14, wherein X² is —C(═O)—.

16. The compound of claim 1, which is a compound of the formula: 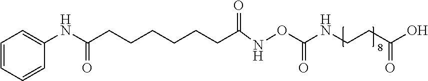
or a pharmaceutically acceptable salt thereof.

17. A pharmaceutical composition comprising:
a compound of any one of claims 1 to 16; and
a protein, wherein the protein is human serum albumin or a protein whose sequence is at least 50% equivalent to that of human serum albumin.

18. The pharmaceutical composition of claim 17, wherein the protein is human serum albumin.

19. The pharmaceutical composition of claim 17 or 18, further comprising a carrier.

20. The pharmaceutical composition of claim 19, wherein the carrier comprises water.

21. The pharmaceutical composition of claim 20, wherein the compound and the protein are non-covalently associated with each other with a binding constant (K_{b}) of at least 10² M⁻¹, or at least 10³ M⁻¹, or at least 10⁴ M⁻¹, or at least 10⁵ M⁻¹.

22. The pharmaceutical composition of any one of claims 19 to 21, wherein the compound and the protein are solvated by the carrier.

23. The pharmaceutical composition of any one of claims 19 to 22, which contains one or more compounds of any one of claims 1 to 16 and one or more proteins, wherein at least 90% by weight, or at least 95% by weight, or at least 97% by weight, or at least 99% by weight, of the compounds in the composition are bound to proteins with a binding constant (K_{b}) of at least 10² M⁻¹, or at least 10³ M⁻¹, or at least 10⁴ M⁻¹, or at least 10⁵ M⁻¹.

24. The pharmaceutical composition of claim 23, wherein at least at least 90% by weight, or at least 95% by weight, or at least 97% by weight, or at least 99% by weight, of the protein-bound particles in the composition have a radius no greater than 5 nm, or no greater than 4 nm, as measured by dynamic light scattering.

25. The pharmaceutical composition of any one of claims 19 to 24, wherein the pharmaceutical composition is suitable for parenteral administration to a mammal, e.g., a human.

26. The pharmaceutical composition of any one of claims 19 to 24, wherein the pharmaceutical composition is suitable for intravenous administration to a mammal, e.g., a human.

27. A pharmaceutical composition comprising:
a compound, which comprises a histone deacetylase inhibiting moiety and a protein binding moiety;
a protein, wherein the protein is human serum albumin or a protein whose sequence is at least 50% equivalent to that of human serum albumin; and
a carrier, which comprises water;
wherein the compound and the protein are non-covalently associated with each other with a binding constant (K_{b}) of at least 10² M⁻¹, or at least 10³ M⁻¹, or at least 10⁴ M⁻¹, or at least 10⁵ M⁻¹; and
wherein the compound and the protein are solvated by the carrier.

28. The pharmaceutical composition of claim 27, wherein the compound is a compound of any one of claims 1 to 16.

29. The pharmaceutical composition of claim 27 or 28, wherein the protein is human serum albumin.

30. The pharmaceutical composition of any one of claims 27 to 29, which contains one or more compounds of any one of claims 1 to 16 and one or more proteins, wherein at least 90% by weight, or at least 95% by weight, or at least 97% by weight, or at least 99% by weight, of the compounds in the composition are bound to proteins with a binding constant (K_{b}) of at least 10² M⁻¹, or at least 10³ M⁻¹, or at least 10⁴ M⁻¹, or at least 10⁵ M⁻¹.

31. The pharmaceutical composition of claim 30, wherein at least at least 90% by weight, or at least 95% by weight, or at least 97% by weight, or at least 99% by weight, of the protein-bound particles in the composition have a radius of no greater than 5 nm, or no greater than 4 nm, as measured by dynamic light scattering.

32. The pharmaceutical composition of any one of claims 27 to 31, wherein the pharmaceutical composition is suitable for parenteral administration to a mammal, e.g., a human.

33. The pharmaceutical composition of any one of claims 27 to 31, wherein the pharmaceutical composition is suitable for intravenous administration to a mammal, e.g., a human.

34. A method of treating cancer, comprising:
administering to a subject a compound of any one of claims 1 to 16 or a composition of any one of claims 17 to 33.

35. The method of claim 34, further comprising administering to a subject an immunotherapy agent.

36. The method of claim 35, wherein administering the immunotherapeutic agent to the subject is carried out concurrently with, or within no more than three days before or after, administering to the subject the compound of any one of claims 1 to 16 or the composition of any one of claims 17 to 33.

37. A method of inducing apoptosis in a cancer cell, comprising:
contacting the cancer cell with a compound of any one of claims 1 to 16 or a composition of any one of claims 17 to 33.

38. A method of inhibiting proliferation of a cancerous tumor, comprising:
contacting the cancerous tumor with a compound of any one of claims 1 to 16 or a composition of any one of claims 17 to 33.

39. Use of a compound of any one of claims 1 to 16 or a composition of any one of claims 17 to 33 as a medicament.

40. Use of a compound of any one of claims 1 to 16 or a composition of any one of claims 17 to 33 for treating cancer.

41. Use of a compound of any one of claims 1 to 16 in the manufacture of a medicament.

42. Use of a compound of any one of claims 1 to 16 in the manufacture of a medicament for treating cancer.
